(19) United States
(12) Patent Application Publication
(10) Pub. No.: US 2004/0236194 A1
(43) Pub. Date: Nov. 25, 2004

(54) Device for measuring at least a physical parameter in a cavity of the organism of a living being

(76) Inventor: Sylvain Meyer, Lausanne (CH)

Correspondence Address: DAVIS & BUJOLD, P.L.L.C., FOURTH FLOOR, 500 N. COMMERCIAL STREET, MANCHESTER, NH, 03101-1151, US

(21) Appl. No.: 10/486,959

(22) PCT Filed: Aug. 14, 2002
(86) PCT No.: PCT/CH02/00444

(30) Foreign Application Priority Data
Aug. 16, 2001 (FR) 01 10844

(51) Int. Cl. A61B 5/07

(52) U.S. Cl. 600/302

(57) ABSTRACT

A device for measuring at least a physical parameter in a cavity of the organism of a living being and for controlling a function of an actuator implanted in the organism comprising a capsule (21) for measuring a physical parameter such as pressure. The capsule (21) contains at least a sensor (11) for measuring the specified parameter, a mechanism for processing signals from the sensor, a storage and power supply mechanism (14). The capsule (21) may also comprise a mechanism for communicating data corresponding to the signals generated by the sensor. The communication mechanism consists of a communication management device and a transducer (19) capable of transmitting ultrasonic signals to a reading equipment arranged outside the living being.

This application is a national stage completion of PCT/C1102/00444 filed c Aug. 14, 2002 which claims priority from French Application Serial No. 01 10844 filed August 16, 2001.

### FIELD OF THE INVENTION

### BACKGROUND OF THE INVENTION

### SUMMARY OF THE INVENTION

### BRIEF DESCRIPTION OF THE DRAWINGS

The invention will now be described, by way of example, with reference to drawings in which:

### DETAILED DESCRIPTION OF THE INVENTION

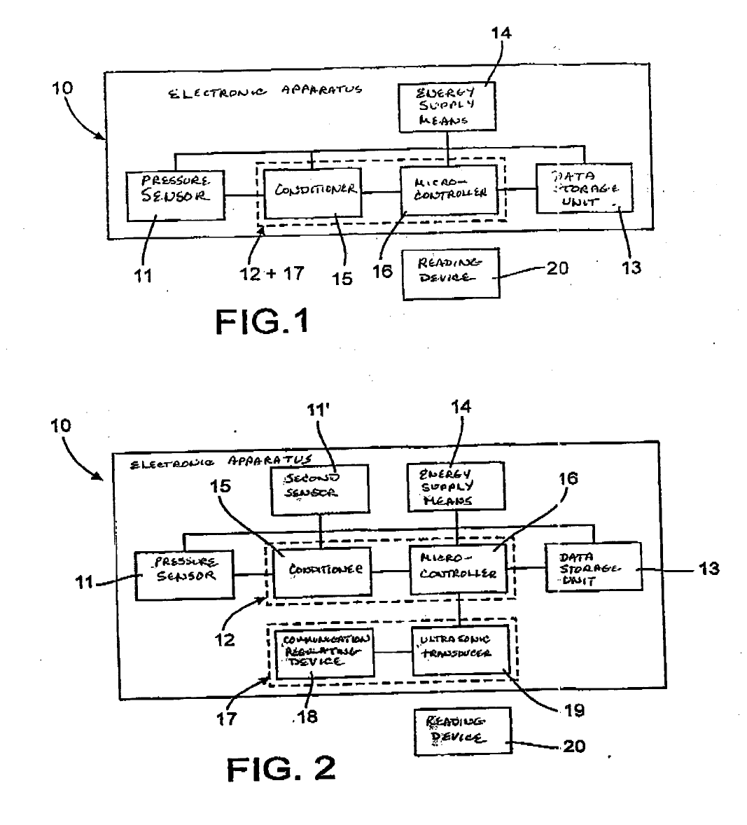

In a first embodiment of the device, an electronic apparatus 10 is housed Inside the capsule 21, as shown schematically in FIG. 1, comprising a pressure sensor 11 for measuring a predetermined parameter, a processing means 12 for processing signals generated by the sensor 11 combined with a means 17 for communicating data corresponding to said signals, a data storage or memory unit 13, and an energy supply means 14.

The energy supply means 14 may consist of a cell, a battery, or a super capacity. It may also consist of a coil capable of supplying all the electronic components by induction. This last variation is of interest when a large or regular supply of energy is required. In this instance, it might not be possible to integrate a source containing sufficient energy into the available space. The ability to remotely recharge the capsule with energy without any contact solves this problem. In general, the electronic components are selected on the basis of particularly low energy consumption, since the space available for energy storage inside the capsule 21 is very limited.,

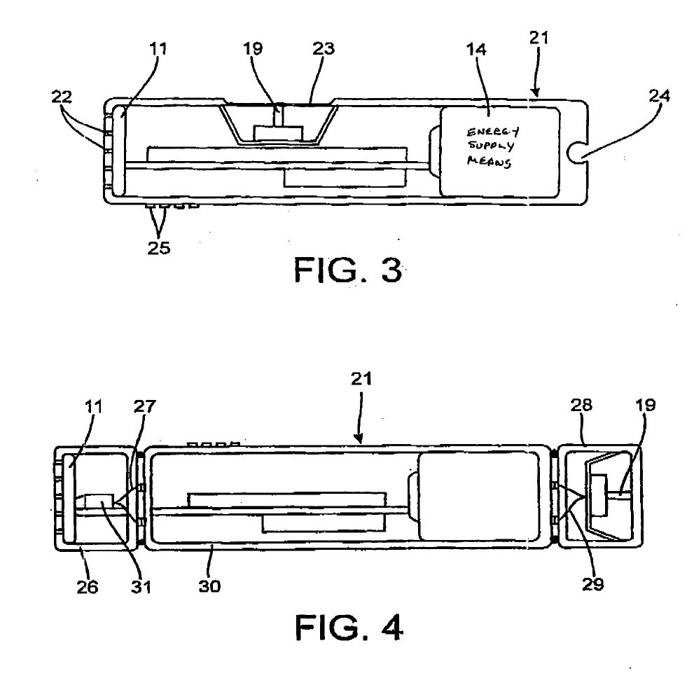

In the example shown in FIG. 3, the sensor 11 is a pressure sensor. The capsule 21 has holes 22 allowing it to measure pressure or variations in pressure. Another role of the sensor 11 is to act as a sealed barrier insulating the inside of the capsule 21 from the outside environment.

The capsule wall comprises one thinner area forming a resonating zone 23 that can be compared to a vibrating membrane. The ultrasonic transducer 19 is located within the capsule 21 in such a way that the resonator, that is, the portion of the ultrasonic transducer 19 which produces vibrations, contacts this zone 23. The shape of the ultrasonic transducer 19 and the v resonating zone 23 define an ultrasonic wave propagating acne which varies in v width and the extent to which it is open depending upon the application.

In this example, the capsule 21 comprises an attachment device 24 to which a wire might be attached, for example (not shown). This allows the capsule 21 to be withdrawn when it Is no longer necessary t) use it.

It is also possible to provide electrical contacts 25 along the periphery of the capsule 21 to transmit the measurement values using a conventional connecting cable (not shown) when the capsule 21 is withdrawn from the patient's body.

This type of embodiment is of particular interest because it is easy to add or remove the ultrasonic transducer 19 and to change the type of sensor without changing the entire device. It is also possible to include a signal preprocessing device 31 in the first module 26 so that no matter what type of sensor is used, the output signal from the first module 26 can be used by the same one, which may be integrated in the third module 30.

The operation of the device of the invention is described below with reference to two examples of its use. In the first example, shown schematically by FIGS. 1 and 3, pressure is measured inside a cavity of a human body, specifically the rectum. Such a measurement is particulary useful when a patient is bedridden and the organism is subjected to particularly high pressure that can cause irreversible lesions. For this purpose, the capsule 21 may be attached to a connecting wire, then introduced inside the rectum. The pressure is measured by the pressure sensor 11, perhaps every 30 seconds. The signals generated by the sensor 1, representing the measured pressure values, are transmitted to the signal processing means 12. The values are first recorded in the data storage unit 13 and then transmitted to the reading device 20 by means of the ultrasonic transducer 19. To do this, the measured values are transformed into coded signals transmitted through the patiant's tissue in the form of ultrasonic waves by the ultrasonic transducer 19. The waves are received by the appropriate reading device 20 which decodes the signals in order to extract pertinent information. This data may then be viewed on a display screen or processed in a processing device such as a computer. The reading device 20 may also be associated with a sensor that produces a visual or auditory alarm when the measured value of a physical parameter exceeds a certain threshold value. With a bedridden individual, exceeding the pressure threshold indicates that intervention is necessary to avoid physiological problems. When measurement is completed, the capsule 11 can be removed from the patient using the connecting wire.

In this embodiment, the data may be transmitted to the reading device 20 located outside the human body, but it may also be transmitted to a device placed within the human body or in contact with it. This device may consist of an actuator such as a forceps, for example, which may remove tissue, or a reservoir of medical products, such as anesthesia, for example, said reservoir having a regulated valve for releasing a predetermined amoun of the substance into the organism. This activator might also be used to initiate the recording of photographs.

The device 10 according to the invention, offers many advantages over prior art devices. In particular, because of the reduced size of the capsule 21, generally less than 10 mm long and 4 mm in diameter, it can be introduced quite easily into the patient's body, either permanently or temporarily, The measurements can either be stored and/or sent to a remote reading device 20. It is also possible to transmit commands to this device from the outside, allowing the measurements to evolve according to reed.

1-20. (Canceled)

21. A device for measuring at least one physical parameter, specifically one or more of pressure and temperature, inside a cavity of a organism of a living being, and for controlling one operation of an apparatus implanted in said organism, the device comprising a capsule (21) implanted in said cavity, said capsule containing at least one sensor (11) for, at least one of, measuring said physical parameter, and for controlling an actuator implanted in said organism, a means (12) for processing the at least one sensor (11), a means (17) for communicating data corresponding to said processed signals, and a memory storage unit (13); and further comprising a reading apparatus (20) located outside the organism of said living being for interpreting and displaying said data corresponding to said signals, characterized in that the said communication means (17) comprises an ultrasonic transducer (19′ for transforming said signals into ultrasonic waves, to transform the data from the capsule (21) in a form of ultrasonic waves to said reading apparatus (20), and for transmitting the data from said reading apparatus (20) to the capsule (21) or from the capsule (21) of the reading apparatus (20) to said actuator.

22. The device according to claim 21, wherein the processing means (12) comprises at least one conditioner (15) and at least one microcontroller (16).

23. The device according to claim 21, further comprises at least one memory storage unit (13) for storing the data processed by the processing means (12) generated by the at least one sensor (11, 11′).

24. The device according to claim 23, wherein the at least one memory storage unit (13) is a non-volatile storage unit.

25. The device according to claim 21, wherein the communication means (17) are distinct from the processing means (12) and comprise a device (18) for regulating communication of the data to said external reading apparatus (20).

26. The device according to claim 21, wherein the transducer (19) is housed in the capsule (21).

27. The device according to claim 21, wherein the capsule (21) comprises a resonating zone (23) located near the transducer (19).

28. The device according to claim 21, wherein the device comprises two or more sensors (11, 11′).

29. The device according to claim 28, wherein at least one of the two or more sensors is a pressure sensor (11).

30. The device according to claim 22, wherein the at least one micro-controller (16) comprises a means for discriminating between the signals originating from the organism of the living being in which the device is implanted and artificially created signals from the outside.

31. The device according to claim 21, further comprises at least two modules (26, 28, 30), one of the modules (26) comprising said sensor (11).

32. The device according to claim 21, wherein the capsule is formed of at least two modules (26, 28, 30), one of the modules (28) comprising said transducer (19).

33. The device according to claim 21, wherein the capsule (21) comprises a means (24) for joining the capsule to a connecting wire.

34. The device according to claim 21, further comprises electrical contacts (25) accessible from the outside of the capsule (21).

35. The device according to claim 31, wherein each elf the at least two modules (26, 28, 30) comprises electrical contacts (27, 29) associated with similar electrical contacts on the adjacent module.

36. The device according to claim 31, wherein each of the at least two modules (26, 28, 30) comprises a means for attatchment to the adjacent module.

37. The device according to claim 21, further comprises an electrical supply means (14).

38. The device according to claim 37, wherein the electrical supply means (14) comprises at least one coil for supplying electrical energy by induction.

39. The device according to claim 21, wherein the capsule has holes (22) for placing the sensor (11) in contact wrth an environment in which the capsule has been placed.

40. The device according to claim 21, wherein said sensor (11) is attached to an outside of the capsule (21).
